(19) United States
(12) Patent Application Publication
(10) Pub. No.: US 2024/0424232 A1
(43) Pub. Date: Dec. 26, 2024

(54) APPARATUS FOR ADMINISTERING MEDICAMENTS AND METHOD FOR MANAGING EXPIRY DATES OF A MEDICAMENT DISPENSER

(71) Applicant: CHIESI FARMACEUTICI S.P.A., Parma (IT)

(72) Inventor: Charles POTTER, PARMA (IT)

(21) Appl. No.: 18/704,036

(22) PCT Filed: Nov. 8, 2022
(86) PCT No.: PCT/EP2022/081136

§ 371 Date: Apr. 23, 2024

(30) Foreign Application Priority Data
Nov. 8, 2021 (EP) 21207001.5

(51) Int. Cl. A61M 15/00; G16H 20/13; G16H 40/63

(52) U.S. Cl. CPC A61M 15/008; G16H 20/13; G16H 40/63; A61M 2205/18; A61M 2205/3306; A61M 2205/6072; A61M 2209/06

(57) ABSTRACT

An apparatus for administering medicaments includes a dispenser and a portable electronic device. An application loaded in the portable electronic device or in a remote location is configured for: receiving and optionally storing data of the dispenser and a date of reading when reading devices read a machine-readable code at the date of reading and, if the machine-readable code has been read for the first time after manufacturing the dispenser, then: calculating and storing an in-use expiry date from the date of reading and an in-use life period and/or starting an in-use expiry counter. An alert is enabled as a function of the in-use expiry date and/or of the in-use life period, so that the portable electronic device issues the alert when the in-use expiry date is reached or approaching or when the in-use life period is elapsed or about to elapse.

### TECHNICAL FIELD OF THE INVENTION

The present invention relates to an apparatus for administering medicaments and to a method for managing expiry dates of a medicament dispenser. In particular, the present invention relates to an improvement of the safety features of medicament hand-held dispensers, such as but not limited to dry powders inhalers DPI or pressurized metered dose inhalers pMDI or soft mist inhalers SMI, to assist the patient to use the medicament correctly.

### BACKGROUND ART

The administering of doses of a medicament from a dispenser containing a multi-dose amount of the medicament is commonly known. The dispenser comprises a dispensing device which is configured for dispensing unit doses of the medicament when the metering device is actuated by a user.

For instance, among the devices available to deliver medicaments to the lungs, multiple-dose type dry powder inhalers (DPI) are widely used. Document WO 2004/012801, by the same Applicant, discloses a dry powder inhaler comprising a container for storing a multi-dose amount of a powdered medicament, a metering member having a dosing recess to be filled with a dose of the powdered medicament and a mouthpiece in communication with an inhalation channel of the powder inhaler. The dry powder inhaler further comprises an inhalation actuated mechanism in such a manner that the inhalation actuated mechanism allows the medicament to be dispensed and inhaled by a user if and when there is an inhalation suction force exerted by the user which exceeds a predetermined level.

Pressurized metered-dose inhalers (pMDIs) to deliver medicaments to the lungs are also widely used. Document EP2890437B1, by the same Applicant, discloses an aerosol inhalation device comprising a housing adapted to receive an aerosol canister containing a pressurised medicament formulation, a mouthpiece portion through which the user inhales and a nozzle block having an orifice which serves to propel the aerosol formulation into the mouthpiece portion.

Document EP3370810B1 discloses an example of soft mist inhaler (SMI) comprising a nebulizer for nebulizing a fluid.

Another kind of multi-dose dry powder inhaler is disclosed in document WO2004011071A1 and comprises a blister strip containing a number of discrete doses of powdered medicament and a mechanism of accessing these doses, comprising either piercing means or means to peel a lid sheet away from a base sheet.

Regardless of the type of the known dispenser, an expiry date of the medicament is generally printed on the packaging of the dispenser or printed on the dispenser or is contained in a machine readable code of a label or tag, like a barcode or a QR code. The expiry date may be printed or included in the machine readable code at the time of manufacturing or packaging the dispenser.

In some circumstances, the multi-dose medicament in the dispenser may be affected by environmental conditions, like moisture and/or temperature and/or light and/or pressure and/or other contamination that may affect properties (e.g. sterility) of the medicament, once the dispenser is in the hands of a user and is used for the first time. For instance, the multi-dose medicament in the housing of the DPI or in the canister of the pMDI or SMI is a quantity of material sufficient to deliver many unit doses but said quantity of material may not be divided into sealed individual doses when contained in the housing. The blisters of the multi-dose dry powder inhalers, as the one disclosed by WO2004011071A1, are not made of a material suitable to protect each single dose from the environmental conditions.

For instance, once removed from its sealed packaging, moisture may affect the drug in a dry powder inhaler (DPI) and/or the drug is no longer sterile and so the product may need to be disposed after a certain period of use, for instance six months after removing from the sealed packaging, even if the dispenser is not empty and the expiry date has not yet been reached.

Also some kinds of metered dose inhalers (MDI) are stored in a sealed packaging after manufacturing and before first use. Other types of metered dose inhalers (MDI), are kept in a fridge during distribution and storage at a pharmacy and when a patient starts to use the device they do not need to keep it in the fridge but at room temperature. The product may then have a three or four months in-use life (from extraction from the fridge) before it should be disposed of.

Those medicaments that can be affected by the environmental conditions, may have therefore two expiry dates: the above mentioned expiry date and an in-use expiry date which is calculated from the first use of the dispenser, i.e. when the dispenser/medicament is first exposed to normal environmental conditions, e.g. when the DPI is removed from its sealed packaging or when the MDI is extracted from the fridge.

The in-use expiry date cannot be printed on the packaging of the dispenser or printed on the dispenser or included in a machine readable code, because during manufacturing it is not known when the patient will first use the product.

Therefore, in the known dispensers, the in-use expiry date must be calculated by the user from an in-use life period written in the instructions for use and the user must keep a note of when he/she started to use the product so that he/she can throw it away after the in-use expiry date.

The risk that the user forgets to keep a note is high and therefore there is a real risk that the medicament will be used after it has lost its potency and/or safety or it may be thrown away when still usable.

Moreover, if the user has multiple inhalers, then he/she needs to be able to identify each one separately and may confuse them with each other.

Document EP3349719B1 discloses a method of counting medicament doses dispensed by an inhaler. WO2012/004298 discloses a drug delivery system comprising control means adapted to store information representing the recommended maximum service lifetime of a reservoir assembly. US2014/0155827 discloses an emergency medicament device, like an auto-injector of epinephrine, comprising a memory and a display to store and display an expiration date of the medicament. EP3348295A1 discloses a medicine injection device capable of managing a term of expiration of a formulation after the start of use of the formulation.

### SUMMARY

It is an object of the present invention to eliminate the above drawbacks of hitherto known dispensers.

Firstly, it is an object of the present invention to provide a system for administering medicaments with an improved safety.

It is also object of the present invention to provide a system that makes easier for the user/patient to take the medicament.

It is also object of the present invention to provide a system that allow the user/patient to order a new prescription in time, thus preventing the risk of running out of the medicament.

At least one of the above objects is substantially achieved by an apparatus for administering medicaments and by a method for managing expiry dates of a medicament dispenser according to one or more of the appended claims and/or of the following aspects.

Aspects of the invention are disclosed in the following.

In accordance with a 1^{st} independent aspect, an apparatus for administering medicaments, comprises:
a dispenser comprising a housing, a dispensing device, optionally a metering device, connected to the housing and a multi-dose amount of a medicament contained in the housing, wherein the dispensing device is configured for dispensing unit doses of said medicament when actuated by a user;
a label or tag comprising a machine readable code comprising or linked to data of the dispenser, wherein the label or tag is fixed to the dispenser and/or contained in or attached to a packaging for the dispenser, wherein the data of the dispenser comprise an in-use life period ΔT_{in_use} of the medicament;
a portable electronic device comprising an electronic control unit and reading devices configured for reading the data of the dispenser from the machine readable code;
an application loaded in the electronic control unit or in a remote location, optionally accessed over a network connection (e.g. a web based application) and configured for performing the following procedure:
   receiving and optionally storing the data of the dispenser and a date of reading Tread when the reading devices read the machine readable code at said date of reading Tread and, if at least one of said data of the dispenser is new indicating that the machine readable code has been read for the first time after manufacturing the dispenser, then:
   calculating and storing an in-use expiry date Texp_{in_use} from the date of reading Tread and the in-use life period ΔT_{in_use} as Texp_{in_use}=Tread+ΔT_{in_use} and/or starting an in-use expiry counter;
   enabling at least one alert as a function of the in-use expiry date Texp_{in_use} and/or of the in-use life period ΔT_{in_use}, so that the portable electronic device issues said at least one alert when the in-use expiry date Texp_{in_use} is reached and/or is approaching or when the in-use life period ΔT_{in_use} is elapsed or is about to elapse.

In a 2^{nd} independent aspect, a method for managing expiry dates of a medicament dispenser, comprises:
reading and optionally storing, through reading devices of a portable electronic device and from a machine readable code of a label or tag fixed to a dispenser of a medicament and/or contained in or attached to a packaging for the dispenser, data of the dispenser and a date of reading Tread; wherein the data of the dispenser comprise an in-use life period ΔT_{in_use} of the medicament; and, if at least one of said data of the dispenser is new indicating that the machine readable code has been read for the first time after manufacturing the dispenser, then:
calculating and storing an in-use expiry date Texp_{in_use} from the date of reading Tread and the in-use life period ΔT_{in_use} as Texp_{in_use}=Tread+ΔT_{in_use} and/or starting an in-use expiry counter;
enabling at least one alert as a function of the in-use expiry date Texp_{in_use} and/or of the in-use life period ΔT_{in_use};
issuing, through the portable electronic device, said at least one alert when the in-use expiry date Texp_{in_use} is reached or is approaching and/or when the in-use life period ΔT_{in_use} is elapsed or is about to elapse.

In a 3^{rd} independent aspect, an application for managing a medicament dispenser is loaded or is configured to be loaded in an electronic control unit of a portable electronic device or in a remote location, optionally accessed over a network connection (e.g. a web based application); said application is configured for performing the following procedure:
receiving and optionally storing data of a dispenser and a date of reading Tread when reading devices of the portable electronic device read a machine readable code at said date of reading Tread, wherein the data of the dispenser comprise an in-use life period ΔT_{in_use} of the medicament contained in the dispenser; and, if at least one of said data of the dispenser is new indicating that the machine readable code has been read for the first time after manufacturing the dispenser, then:
calculating and storing an in-use expiry date Texp_{in_use} from the date of reading Tread and the in-use life period ΔT_{in_use} as Texp_{in_use}=Tread+ΔT_{in_use} and/or starting an in-use expiry counter;
enabling at least one alert as a function of the in-use expiry date Texp_{in_use} and/or of the in-use life period ΔT_{in_use}, so that the portable electronic device issues said at least one alert when the in-use expiry date Texp_{in_use} is reached and/or is approaching or when the in-use life period ΔT_{in_use} is elapsed or is about to elapse.

The invention helps ensure the user/patient is not using the medicament after the in-use expiry date. Indeed, only when the dispenser is first used (e.g. removed from the packaging) the in-use expiry counter/clock and the countdown to the in-use expiry date will be started. This way, the invention prevents the user/patient to take the medicament after said medicament has passed the in-use expiry date.

Indeed, after the in-use expiry date the potency of the medication may not be within specification and therefore the patients may not get an adequate dose of medication to treat their illness. Therefore, the product should be thrown away at the end of the in-use expiry even if there are doses remaining.

The invention is useful in particular but not exclusively if the medicament is prescribed as a “reliever” or “as required” product that the patient uses only when he/she feels breathless. In this situation, it is quite difficult for the patient to know how long the product should last. In these circumstances it is far more likely that the device will reach the in-use expiry and still have doses remaining.

The invention provides also further advantages and technical effects.

For instance, the invention makes easier for the user/patient to take the medicament and/or allows the user/patient to order a new prescription in time, thus preventing the risk of running out of the medicament.

Further aspects of the invention are disclosed in the following.

In a 4th aspect according to at least one of the preceding aspects, if said data of the dispenser are new data indicating that the machine readable code has been read for the first time after manufacturing the dispenser, the date of reading Tread is recorded and/or labeled as date of first use of the dispenser after manufacturing (i.e. when the dispenser/medicament is first exposed to normal environmental conditions, e.g. room temperature and/or moisture and/or light and/or pressure and/or other contamination). Optionally, the date of reading Tread or date of first use is used for commercial reasons to understand the supply chain better by knowing the time taken from manufacture to reach the patient in a specific country.

In a 5th aspect according to at least one of the preceding aspects, enabling at least one alert comprises: enabling a final alert.

In a 6th aspect according to the preceding aspect, issuing said at least one alert comprises: issuing the final alert when the in-use expiry date Texp_{in_use} is reached and/or when the in-use life period ΔT_{in_use} is elapsed.

In a 7th aspect according to at least one of the preceding aspects, enabling at least one alert comprises: enabling at least one pre-alert.

In an 8th aspect according to the preceding aspect, issuing said at least one alert comprises: issuing the at least one pre-alert before reaching the in-use expiry date Texp_{in_use} and/or before that the in-use life period ΔT_{in_use} is elapsed.

This enables alerts to be given to the user/patient when the product is reaching the end of its in-use life.

In a 9th aspect according to at least one of the preceding aspects, the data of the dispenser further comprise an expiry date Texp.

In a 10th aspect according to the preceding aspect, the method comprises, or the application is configured for, comparing the expiry date Texp with the in-use expiry date Texp_{in_use} and issuing a further alert if the in-use expiry date Texp_{in_use} occurs after the expiry date Texp and/or if the in-use life period ΔT_{in_use} elapses after the expiry date Texp.

In this case, the user/patient may immediately return the dispenser to the pharmacy and get another.

In a 11th aspect according to at least one of the preceding aspects, the data of the dispenser comprise a unique identifier of the dispenser.

In a 12th aspect according to the preceding aspect, the method comprises, or the application is configured for, associating the unique identifier to the date of reading Tread and/or to the in-use expiry date Texp_{in_use} and/or to the in-use life period ΔT_{in_use} to allow managing a plurality of dispensers.

In a 13th aspect according to at least one of the preceding aspects, the method comprises or the application is configured for performing the following procedure: if the data of the dispenser are not new (i.e. identified as already read by the portable electronic device, e.g. are already present in the electronic control unit or in the remote location), then showing, through output devices of the portable electronic device, a status indicating that the machine readable code has already been read. If the patient has more than one dispenser then the machine readable code can be read again to ensure the right dispenser is being disposed of (i.e. it will recognise that a date of first use has already been stored/labeled for that dispenser).

In a 14th aspect according to at least one of the preceding aspects, the data of the dispenser comprise further information on the dispenser and medicament, e.g. drug, dose, manufacturing site, batch number.

In a 15th aspect according to at least one of the preceding aspects, the method comprises or the application is configured for performing the following procedure: if the data of the dispenser are not new (i.e. identified as already read by the portable electronic device, e.g. are already present in the electronic control unit or in the remote location), then showing, through output devices of the portable electronic device, a remaining in-use life period ΔTᵣₑₘₐᵢₙ.

In a 16th aspect according to at least one of the preceding aspects, in addition to enabling at least one alert, the method comprises or the application is configured for performing the following procedure: enabling further notification and or links, like notifications of when to take the drug, notifications of when the product should be empty if the patient has taken all of their prescribed doses (this feature may enable the patient to understand how compliant they are with adhering to the prescription instructions), instructions for use, links to videos of how to use the device, disposal instructions, etc.,

In a 17th aspect according to at least one of the preceding aspects, the method comprises or the application is configured for performing the following procedure:
receiving or selecting a parameter reflecting a region or country where the portable electronic device is located and/or where a user/patient of the dispenser lives and/or a time of year or season;
choosing, among the data of the dispenser, the in-use life period ΔT_{in_use} for that region or country and/or that time of year or season.

In a 18th aspect according to at least one of the preceding aspects, the portable electronic device comprises a radio receiver and the method comprises or application is configured for performing the following procedure:
receiving from the radio receiver a parameter reflecting a region or country where the portable electronic device is located and/or a time of year or season;
choosing, among the data of the dispenser, the in-use life period ΔT_{in_use} for that region or country and/or that time of year or season.

In a 19th aspect according to the previous aspect, the radio receiver is a GPS receiver or a receiver of mobile network (e.g. 3G, 4G, 5G) or a Bluetooth receiver or a Wi-Fi receiver.

Since in-use life period may depend on the region or country and/or time of year or season (e.g. due to temperature and or humidity differences; some countries/regions are very hot in summer and much cooler in winter), this further function ensures the expiry is correct for the region or country of use and/or time of year or season.

In a 20th aspect according to at least one of the preceding aspects, reading is performed after removing for the first time the dispenser from a sealed packaging or from a controlled atmosphere environment; optionally the sealed packaging is a moisture-protective pouch.

In a 21st aspect according to at least one of the preceding aspects, the dispenser is a hand-held or portable dispenser, optionally an inhaler.

In a 22nd aspect according to the preceding aspect, the inhaler is a dry powder inhaler and the medicament is a powdered medicament; optionally, the multi-dose amount of the medicament contained in the housing is a single quantity of material not divided into sealed individual doses; or, optionally, the multi-dose amount of the medicament contained in the housing comprises a number of discrete doses of powdered medicament contained in blisters.

In a 23rd aspect according to the preceding aspect, reading is performed after removing for the first time the dispenser from a sealed packaging.

In a 24th aspect according to the preceding aspect 22 or 23, the in-use life period ΔT_{in_use} is between five and seven months, optionally of six months.

In a 25th aspect according to aspect 20, the inhaler is a metered dose inhaler (MDI), optionally a pressurized metered dose inhaler (pMDI), or a soft mist inhaler (SMI) and the medicament is a suspension or solution formulation, preferably a solution. In a 26th aspect according to the preceding aspect, reading is performed after removing for the first time the dispenser from a fridge.

In a 27th aspect according to the preceding aspect, the in-use life period ΔT_{in_use} is between two and five months, optionally of three or four months.

In a 28th aspect according to any of the preceding aspects, the medicament is a mono-component or multi-component formulation.

In a 29th aspect according to at least one of the preceding aspects, the label or tag comprises an optical code, like a QR code or a bar code, and the the reading devices comprise an optical reader, like a camera.

In a 30th aspect according any of the preceding aspects 1 to 28, the label or tag is a tag RFID (Radio-frequency identification) and the reading devices comprise an RFID reader or the label or tag is a beacon BLE (Bluetooth low energy) and the reading devices comprise a BLE reader; optionally said label or tag also incorporates a “write function” and the portable electronic device comprises writing devices configured to write in said label or tag.

In a 31st aspect according to any of the preceding aspects, the portable electronic device is a smartphone.

In a 32nd aspect according any of the preceding aspects, the data of the dispenser are contained in the machine readable code and/or in the portable electronic device.

In a 33rd aspect according to the preceding aspect, when the reading devices read the machine readable code at said date of reading Tread, the method comprises or the application is configured for performing the following procedure: reading the data of the dispenser from the machine readable code.

In a 34th aspect according to any of the preceding aspects 1 to 31, the data of the dispenser are contained in a remote location, optionally accessed over a network connection (e.g. a remote database).

In a 35th aspect according to the preceding aspect, when the reading devices read the machine readable code at said date of reading Tread, the method comprises or the application is configured for performing the following procedure:
reading an address of the remote location from the machine readable code;
connecting to the remote location and retrieving said data of the dispenser from the remote location;
optionally storing said data of the dispenser in the portable electronic device or in the label or tag if said label or tag also incorporates a “write function”, such as a tag RFID or a beacon BLE.

In a 36th aspect according to any of the preceding aspects, in order to discern if the data of the dispenser are new data, the method comprises or the application is configured for performing the following procedure: comparing at least one of the data of the dispenser with at least one of the data already stored of other dispensers previously read.

In a 37th aspect according to the preceding aspect and to aspect 11 or 12, it is provided for comparing the unique identifier of the dispenser with unique identifiers already stored of other dispensers previously read.

In a 38th aspect according to any of the preceding aspects, the dispenser comprises a dose counting unit configured to count a number of unit doses dispensed. The dose counting unit is configured to be actuated and incremented each time the dispensing device is actuated.

In a 39th aspect according to any of the preceding aspects, the method comprises or the application is configured for performing the following procedure:
receiving a medical prescription comprising a number of doses per unit time to be taken by the user and an overall assumption time ΔTₐₛₛᵤₘ or a total number of doses to be taken by the user;
considering or calculating the overall assumption time ΔTₐₛₛᵤₘ and comparing said overall assumption time ΔTₐₛₛᵤₘ with the in-use expiry date Texp_{in_use} and/or the in-use life period ΔT_{in_use};
issuing an additional alert if the in-use expiry date Texp_{in_use} occurs before the end of the overall assumption time ΔTₐₛₛᵤₘ and/or if the in-use life period ΔT_{in_use} elapses before the overall assumption time ΔTₐₛₛᵤₘ.

In a 40th aspect according to any of the previous aspects, the in-use expiry date Texp_{in_use} is stored in the portable electronic device or in a remote location, optionally accessed over a network connection (e.g. a remote database), or in the label or tag if said label or tag also incorporates a “write function”, such as a tag RFID or a beacon BLE.

In a 41st aspect according to any of the previous aspects, if the label or tag is a beacon (e.g. BLE), then it could automatically emit a signal when it is first used. This signal may be picked up in a home environment or home hub which could store the in-use expiry date Texp_{in_use} in a remote location.

In a 42^{nd} aspect according to any of the previous aspects, the method comprises or the application is configured for adjusting the date of reading Tread or date of first use and/or to manually enter, through the portable electronic device, the date of reading or date of first use. This may be useful if the user forgets to read the machine readable code when the dispenser is first used.

Further features will appear in greater detail in the detailed description of preferred, but not exclusive, embodiments of an apparatus for administering medicaments and a method for managing expiry dates of a medicament dispenser according to the present invention.

### DESCRIPTION OF THE DRAWINGS

This description will be set out below with reference to the attached drawings, which are provided for indicative and non-limiting purposes, in which:

FIG. 1 shows an apparatus for administering medicaments according to the present invention;

FIG. 2 shows a schematic cross section of one element of the apparatus of FIG. 1;

FIG. 3 shows a different embodiment of the element of FIG. 3;

FIG. 4 shows a schematic cross section of the element of FIG. 3;

FIG. 5 is a schematic representation of part of the apparatus of FIG. 1; and

FIG. 6 is a flow chart of a method according to the invention.

### DETAILED DESCRIPTION

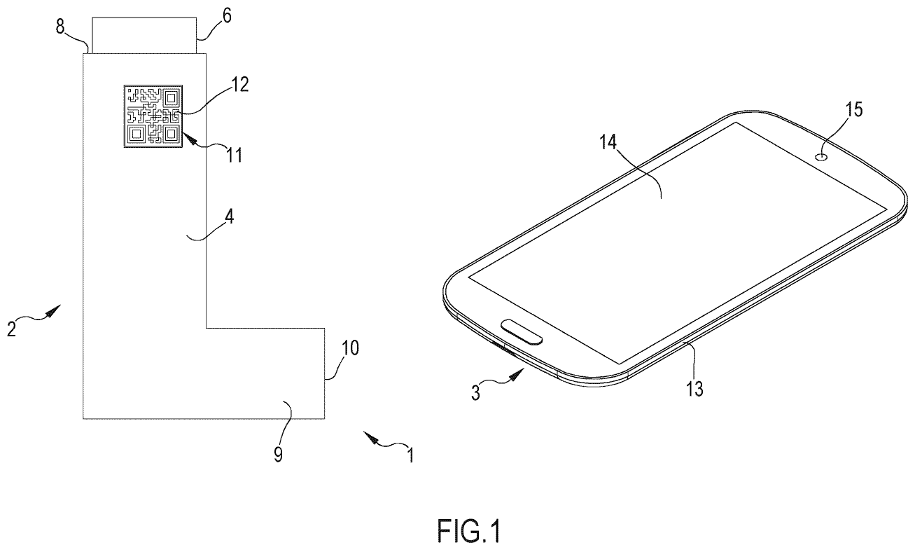

With reference to the appended drawings, FIG. 1 shows an apparatus for administering medicaments. The apparatus 1 comprises a pressurized metered dose inhaler (pMDI) 2, i.e. a portable or hand-held medicament dispenser, and a smartphone 3, i.e. a portable electronic device.

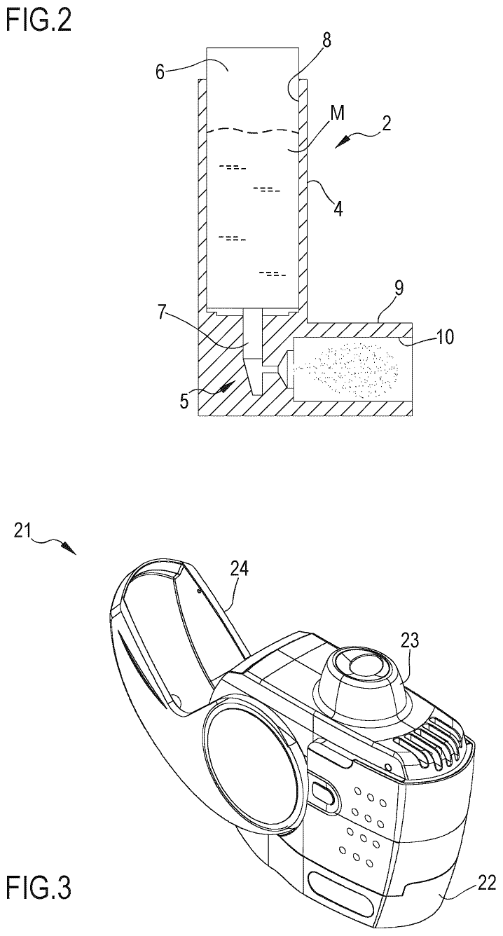

The pressurized metered dose inhaler 2 is known per se and comprises a housing 4 provided with a metering device 5 mounted or located in the housing 4. As better shown in FIG. 2, a reservoir 6 containing a suitable medicament M (e.g. a mono-component or multi-component formulation) and comprising a valve 7 is placed in the housing 4 through an aperture 8 of the housing 4. The reservoir 6 may be disposable. The housing 4 is also provided of a mouthpiece 9 delimiting a mouth 10 for dispensing the medicament M ejected through an orifice of the metering device 5.

The medicament M in the reservoir 6 is a multi-dose amount of medicament M and the metering device 5 allows to dispense a unit dose of said medicament M each time a user presses the reservoir 6 against the housing 4.

The pressurized metered dose inhaler 2 may also comprise a dose counting unit, not shown in the drawings, which is configured to be actuated and incremented or decremented each time the metering device 5 is actuated to count a number of unit doses dispensed.

A pressurized metered dose inhaler 2 of this kind is for instance disclosed in document EP2890437B1 by the same Applicant.

A tag 11 comprising a QR code 12 (i.e. a machine readable code) is applied to an exterior surface of the housing 4. The QR code comprises or is linked to data of the pressurized metered dose inhaler 2 as will be explained hereafter.

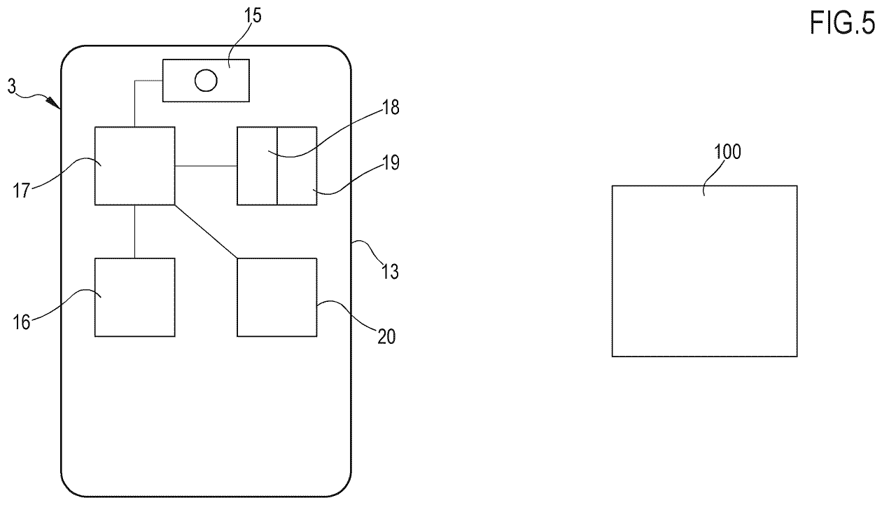

The smartphone 3 is also per se known and comprises a casing 13 with a display 14 and contains (FIG. 5) a camera 15 (reading device), a memory 16, an electronic control unit 17, a radio receiver 18, a radio transmitter 19 and a GPS receiver 20. The electronic control unit 17 is operationally connected to the camera 15, to the memory 16, to the radio receiver 18, to the radio transmitter 19 and to the GPS receiver 20 to properly manage the operations of the smartphone 3.

The radio receiver 18 and the radio transmitter 19 are configured to communicate with a cellular or mobile network (e.g. 3G, 4G or 5G network).

The GPS receiver is configured to receive signals from satellites of the GPS system. The smartphone 3 may be also provided with a Bluetooth transmitter/receiver and a Wi-Fi transmitter/receiver, not shown in the annexed figures.

The apparatus 1 further comprises a software application (App) loaded in the smartphone 3, e.g. stored in the memory 16 of the smartphone 3, and the electronic control unit 17 is configured to run said application and also to perform the method according to the invention.

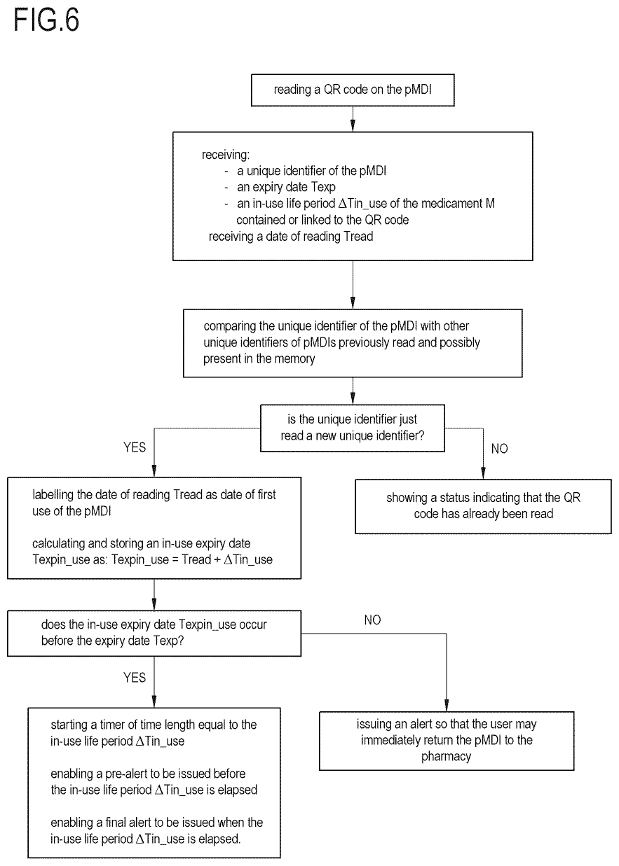

Referring to the flowchart of FIG. 6, the application is configured to read, through the camera 15, the QR code 12 and to receive and store the data of the pressurized metered dose inhaler 2 (pMDI) contained or linked to the QR code.

For instance, in one embodiment, the data of the pMDI may be contained in the QR code or, in another embodiment, said data may be contained in a web-accessible remote database 100 and the QR code contains a web address of said remote database 100, so that, when the user points the camera of the smartphone 3 towards the QR code while the application is running, the application retrieves said data from the remote database 100 and may save them in the memory 16.

The data of the pMDI comprise an in-use life period ΔT_{in_use} of the medicament M, i.e. a period starting from the hypothetical first use of the pMDI, when the medicament M is first exposed to normal environmental conditions, e.g. room temperature and/or moisture and/or light and/or pressure and/or non-sterile conditions. Since pMDIs are kept in a fridge (i.e. a controlled atmosphere environment) during distribution and storage at a pharmacy, the in-use life period ΔT_{in_use} will typically start from the extraction from the fridge when the product is picked up from a pharmacy. The in-use life period ΔT_{in_use} of a pMDI is known to the manufacturer, depends on the formulation of the medicament M and may be for instance three or four months, or even longer. The in-use life period ΔT_{in_use} is, of course, only guaranteed if the user respects the recommendations for use and storage of the product. The in-use life period ΔT_{in_use} is known and part of the data of the pMDI but an in-use expiry date Texp_{in_use} calculated from the hypothetical first use is unknown when the pMDI is manufactured and cannot be part of the data of the pMDI.

The data of the pMDI typically further include a unique identifier of the pMDI, e.g. a serial number.

The data of the pMDI typically further include an expiry date Texp, i.e. an expiry date calculated from the date of manufacturing of the pMDI. Since the date of manufacturing is known, the expiry date Texp is part of the data of the pMDI in the QR code or in the remote database 100. The expiry date Texp is typically the expiry date of the medicament M if said medicament M is kept safe from normal environmental conditions, e.g., if the pMDI is kept in the fridge or sealed in a packaging or pouch during distribution and storage at a pharmacy. Also this expiry date Texp is known to the manufacturer and depends on the formulation of the medicament M.

According to embodiments, the database 100 includes a plurality of expiry dates Texp and/or in-use life periods ΔT_{in_use}, each linked to one or more geographical regions or countries, since the expiry date Texp and the in-use life period ΔT_{in_use} may depend on the region or country due, e.g., to temperature and or humidity differences.

The application is configured for receiving a parameter reflecting the region or country where the smartphone 3 is located and/or the user lives and choosing the expiry date Texp and/or the in-use life period ΔT_{in_use} for that region or country. Said parameter reflecting the region or country may be entered by the user or retrieved by the application from the cellular or mobile network and/or from the GPS system. The application may also be configured for receiving a parameter reflecting the time of year or season when the dispenser is used, since some countries/regions are very hot in summer and much cooler in winter and the climatic conditions greatly affect the medicaments.

The data of the pMDI may also include further information and/or links via the application (e.g. web addresses) to retrieve such information, such as e.g. drug, dose, manufacturing site, batch number, instructions for use, videos of how to use the device, disposal instructions, etc.,

The application is further configured for:
receiving and optionally storing a date of reading Tread, i.e. the date on which the QR code is read;
comparing the unique identifier of the pMDI with other unique identifiers of pMDIs previously read and possibly present in the memory 16.

If the unique identifier just read is the first read or is different from the other unique identifiers, i.e. it is a new unique identifier indicating that the QR code has been read for the first time after manufacturing the pMDI, then:
labeling and storing the date of reading Tread as date of first use of the pMDI after manufacturing associated to the unique identifier;
calculating and storing an in-use expiry date Texp_{in_use} from the date of reading Tread and the in-use life period ΔT_{in_use} as “Texp_{in_use}=Tread+ΔT_{in_use}” and/or starting an in-use expiry counter, i.e. a timer of time length equal to the in-use life period ΔT_{in_use};
comparing the expiry date Texp with the in-use expiry date Texp_{in_use} and, if the in-use expiry date Texp_{in_use} occurs after the expiry date Texp and/or if the in-use life period ΔT_{in_use} elapses after the expiry date Texp then issuing, through the smartphone 3, an alert (e.g. an audible or visual signal or vibration) so that the the user may immediately return the pMDI to the pharmacy and get another, otherwise:
enabling a pre-alert so that said pre-alert may be issued through the smartphone 3 (e.g. a message, an audible or visual signal or vibration) before reaching the in-use expiry date Texp_{in_use} and/or before that the in-use life period ΔT_{in_use} is elapsed (e.g. some weeks or days before to alert the user when the product is reaching the end of its in-use life so a new product can be prescribed by a doctor and picked up from a pharmacy before expiry of the old product);
enabling a final alert so that said final alert may be issued when the in-use expiry date Texp_{in_use} is reached and/or when the in-use life period ΔT_{in_use} is elapsed.

The application may also be configured for adjusting the date of reading Tread or date of first use and/or to manually enter, through the portable electronic device 3, the date of reading Tread or date of first use and this function may be useful if the user forgets to read the machine readable code 12 when the dispenser 2 is first used and/or removed from its packaging.

The application may be also configured to enable further notification and or links. The application may be further configured for receiving a medical prescription for the user/patient, wherein said medical prescription comprises a number of doses per unit time to be taken by the user (e.g. 2 doses per day) and an overall assumption time ΔTₐₛₛᵤₘ (e.g. 90 days) or a total number of doses to be taken by the user (e.g. 180 doses).

The application is also configured to consider or calculate the overall assumption time ΔTₐₛₛᵤₘ and to compare the overall assumption time ΔTₐₛₛᵤₘ with the in-use expiry date Texp_{in_use} and/or with the in-use life period ΔT_{in_use}.

The application is also further configured to issue an additional alert through the smartphone 3 if the in-use expiry date Texp_{in_use} occurs before the end of the overall assumption time ΔTₐₛₛᵤₘ and/or if the in-use life period ΔT_{in_use} elapses before the overall assumption time ΔTₐₛₛᵤₘ. This way, if the pMDI contains a very large number of unit doses and the in-use life period ΔT_{in_use} is very short, the user may be warned that he will not be able to use all the medicament.

The application, if desired, can also provide notifications when the user should use their product. These would be entered by the user at the preferred times of day when the patient is prescribed to take their medication e.g. 8.00 am in the morning and 6.00 μm in the evening.

If the unique identifier just read is the same as one of the other unique identifiers in the memory 16, i.e. it is not a new unique identifier indicating that the QR code has already been read, then:
showing, through the display 14 of the smartphone 3, a status (a message, an image, etc.) indicating that the QR code has already been read and a remaining in-use life period ΔTᵣₑₘₐᵢₙ of the pMDI.

The application allows therefore to manage a plurality of pMDIs or other inhalers or dispensers and ensures the right inhaler is being disposed of when the final alert is issued.

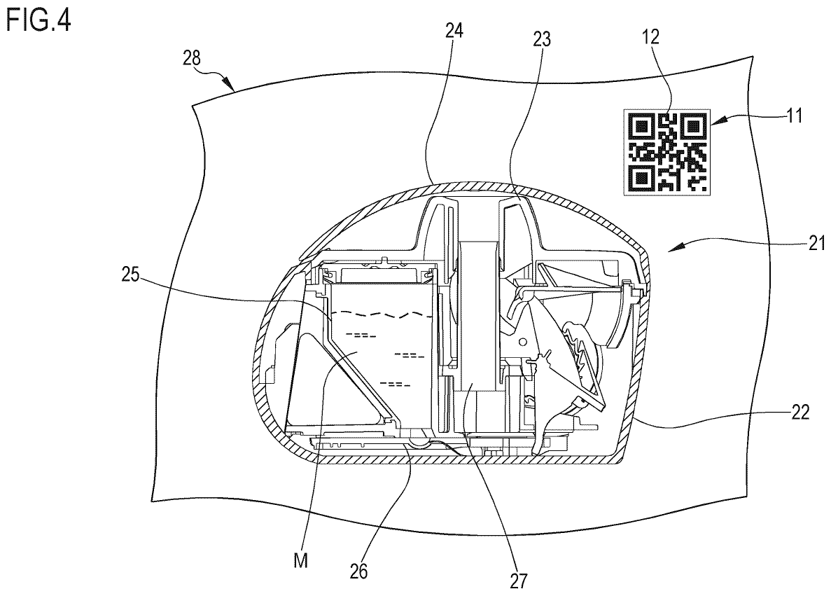

FIGS. 3 and 4 show another kind of inhaler, i.e. a dry powder inhaler (DPI) 21 which may be, for instance, the dry powder inhaler disclosed in document WO 2004/012801, by the same Applicant. Also this dry powder inhaler 21 may be used with the application and the smartphone 3 described above and in the same manner as described above.

The dry powder inhaler 21 shown in FIGS. 3 and 4 comprises a housing 22 delimiting a mouthpiece 23 and provided with a mouthpiece cover 24 configured to open or close the mouthpiece 23. A container 25 inside the housing 22 stores a multi-dose amount of a powdered medicament M (for instance a mono-component or multi-component formulation). A metering member 26 having a dosing recess to be filled with a dose of the powdered medicament M can be placed in communication with an inhalation channel 27 and the mouthpiece 23. The dry powder inhaler 21 further comprises an inhalation actuated mechanism configured in such a manner that the inhalation actuated mechanism allows the medicament M to be dispensed and inhaled by the user if and when there is an inhalation suction force exerted by the user which exceeds a predetermined level.

During distribution and storage at a pharmacy, the dry powder inhaler 21 is generally contained in a sealed packaging 28 (FIG. 4) to prevent to be affected by the environmental conditions. The sealed packaging may be, for instance, a moisture-protective foil laminate pouch with a desiccant that should be discarded when the pouch is opened.

As shown in FIG. 4, the tag 11 comprising the QR code 12 is attached to the sealed packaging 28 and the user reads the QR code through the camera 15 of the smartphone 3 when extracts the dry powder inhaler 21 from the sealed packaging 28 on the first use. In another embodiment, the QR code may be printed on the housing 22 of the dry powder inhaler 21.

The shelf-life expiry date Texp_{shelf} for the dry powder inhaler 21 depends on the formulation and may be eighteen months.

Also the in-use expiry date Texp_{in_use} depends on the formulation and may be six months.

According to other embodiments of the invention, the apparatus 1 may comprise other kind of dispensers comprising a housing, a dispensing device connected to the housing and a multi-dose amount of a medicament contained in the housing (i.e., the multi-dose medicament in the housing is a quantity of material sufficient to deliver many unit doses but said quantity of material is not necessarily divided into sealed individual doses when contained in the housing), wherein the dispensing device is configured for dispensing unit doses of said medicament when actuated by the user. For instance, the dispenser may be an MDI provided with pre filled blisters, like the one disclosed in document WO2004011071A1 or a soft mist inhaler (SMI) as disclosed in document EP3370810B1.

According to embodiments, the application may be not loaded in the electronic control unit but is a web based application accessed over a network connection.

Other tags or labels provided with a machine readable code may be employed instead of the QR code, for instance, a bar code or a tag RFID (Radio-frequency identification) or a beacon BLE (Bluetooth low energy). Accordingly, the reader will be an optical reader (like the camera 15) or a RFID reader or a BLE reader. Since these kind of tags (RFID reader or a BLE reader) may incorporate a “write function”, the data of the dispenser 2 and/or the in-use expiry date Texp_{in_use} may be stored in the tags. For this purpose, the portable control unit 3 comprises writing devices to write the data of the dispenser 2 and/or the in-use expiry date Texp_{in_use} in the tags. If the label or tag is a beacon BLE, then it could automatically emit a signal when it is first used. This signal may be picked up in a home environment (e.g. Alexa™) or home hub which could store the in-use expiry date Texp_{in_use} in the cloud.

The tag or label with the machine readable code may be attached on the housing of the dispenser, like the pMDI 2 disclosed above, on the packaging, like the DPI 21 disclosed above, but also on a sheet inserted in the packaging 28 of the dispenser or fixed to the disposable reservoir 6 of the pMDI 2. If components of a DPI are reused then the machine readable code will be attached to the disposable, medicament containing housing.

### LIST OF ELEMENTS

apparatus 1
pressurized metered dose inhaler (pMDI) 2
smartphone 3
housing 4
metering device 5
reservoir 6
valve 7
aperture 8
mouthpiece 9
mouth 10
tag 11
QR code 12
casing 13
display 14
camera 15
memory 16
electronic control unit 17
radio receiver 18
radio transmitter 19
GPS receiver 20
dry powder inhaler (DPI) 21
housing 22
mouthpiece 23
mouthpiece cover 24
mouthpiece 23
container 25
metering member 26
inhalation channel 27
sealed packaging 28

1.-15. (canceled)

16. An apparatus for administering medicaments, comprising:
a dispenser comprising a i) housing, ii) a dispensing device connected to the housing and iii) a multi-dose amount of a medicament contained in the housing, wherein the dispensing device is configured for dispensing unit doses of said medicament when actuated by a user;
a label or tag comprising a machine-readable code comprising or linked to data of the dispenser, wherein the label or tag is i) fixed to the dispenser and/or iia) contained in or iib) attached to a packaging for the dispenser, wherein the data of the dispenser comprise an in-use life period (ΔT_{in_use}) of the medicament;
a portable electronic device comprising an electronic control unit and reading devices configured for reading the data of the dispenser from the machine-readable code;
an application loaded in the electronic control unit or in a remote location and configured for:
receiving and optionally storing the data of the dispenser and a date of reading (Tread) when the reading devices read the machine-readable code at said date of reading;
if at least one of said data of the dispenser is new, thus indicating that the machine-readable code has been read for the first time after manufacturing the dispenser, then:
calculating and storing an in-use expiry date (Texp_{in_use}) from the date of reading and the in-use life period as Texp_{in_use}=Tread+ΔT_{in_use} and/or starting an in-use expiry counter; and
enabling at least one alert as a function of the in-use expiry date and/or of the in-use life period, so that the portable electronic device issues said at least one alert i) when the in-use expiry date is reached or approaching or ii) when the in-use life period is elapsed or about to elapse.

17. The apparatus of claim 16, wherein the data of the dispenser further comprise an expiry date and the application is configured for comparing the expiry date with the in-use expiry date and to issue a further alert i) if the in-use expiry date occurs after the expiry date and/or ii) if the in-use life period elapses after the expiry date.

18. The apparatus of claim 16, wherein
the data of the dispenser comprise a unique identifier of the dispenser, and
the application is configured for associating the unique identifier to at least one of i) the date of reading, ii) the in-use expiry date, and iii) the in-use life period, to allow managing of a plurality of dispensers.

19. The apparatus of claim 16, wherein the application is configured for:
if the data of the dispenser are not new, showing, through output devices of the portable electronic device, i) a status indicating that the machine-readable code has already been read and/or ii) a remaining in-use life period.

20. The apparatus of claim 16, wherein the portable electronic device comprises a radio receiver and the application is configured for:
receiving from the radio receiver a parameter indicating i) a region or country where the portable electronic device is located and/or ii) a time of year or season; and
choosing, among the data of the dispenser, the in-use life period for said region or country and/or said time of year or season.

21. The apparatus of claim 16, wherein the dispenser is an inhaler;

22. The apparatus of claim 21, wherein the inhaler is a dry powder inhaler and the medicament is a powdered medicament.

23. The apparatus of claim 21, wherein the inhaler is a metered dose inhaler, optionally a pressurized metered dose inhaler.

24. The apparatus of claim 21, wherein the inhaler is a soft mist inhaler and the medicament is a suspension or solution formulation.

25. The apparatus of claim 16, wherein the label or tag comprises a QR code, the portable electronic device is a smartphone and the reading devices comprise a camera.

26. A method for managing expiry dates of a medicament dispenser, comprising:
reading and optionally storing, through reading devices of a portable electronic device and from a machine-readable code of a label or tag i) fixed to a dispenser of a medicament and/or iia) contained in or iib) attached to a packaging for the dispenser, data of the dispenser and a date of reading (Tread); wherein the data of the dispenser comprise an in-use life period (ΔT_{in_use}) of the medicament;
if at least one of said data of the dispenser is new, thus indicating that the machine-readable code has been read for the first time after manufacturing the dispenser, then:
calculating and storing an in-use expiry date (Texp_{in_use}) from the date of reading and the in-use life period as Texp_{in_use}=Tread+ΔT_{in_use} and/or starting an in-use expiry counter;
enabling at least one alert as a function of the in-use expiry date and/or of the in-use life period; and
issuing, through the portable electronic device, said at least one alert when the in-use expiry date is reached or is approaching and/or when the in-use life period is elapsed or is about to elapse.

27. The method of claim 26, comprising:
comparing an expiry date, which is part of the data of the dispenser, i) with the in-use expiry date and/or ii) with the in-use life period and
issuing a further alert i) if the in-use expiry date occurs after the expiry date and/or ii) if the in-use life period elapses after the expiry date.

28. The method of claim 26, comprising:
associating a unique identifier of the dispenser to the date of reading and/or to the in-use expiry date and/or to the in-use life period to allow managing a plurality of dispensers.

29. The method of claim 26, comprising:
if the data of the dispenser are not new, showing, through output devices of the portable electronic device, a status indicating that the machine-readable code has already been read.

30. The method of claim 26, comprising:
if the data of the dispenser are not new, showing, through output devices of the portable electronic device, a remaining in-use life period.

31. The method of claim 26, further comprising:
receiving or selecting a parameter indicating i) a region or country where the portable electronic device is located and/or where a user of the dispenser lives and/or ii) a time of year or season; and
choosing, among the data of the dispenser, the in-use life period for said region or country and/or said time of year or season.

32. The method of claim 26, wherein
the dispenser is a dry powder inhaler and the medicament is a powdered medicament; and
reading is performed after removing for the first time the dry powder inhaler from a sealed packaging.

33. The method of claim 26, wherein
the dispenser is a metered dose inhaler, optionally a pressurized metered dose inhaler, or a soft mist inhaler;
the medicament is a suspension or solution formulation; and
reading is performed after removing for the first time the metered dose inhaler or the soft mist inhaler from a fridge or from a sealed packaging
